SEQUENCE LISTING (10 sequences)

SEQ ID NO: 1 - protein, 15 aa, Artificial Sequence
  Description of Artificial Sequence Synthetic peptide
GTPGPQGIAGQRGVV

SEQ ID NO: 2 - protein, 9 aa, Artificial Sequence
  Description of Artificial Sequence Synthetic peptide
GPQGIAGQR

SEQ ID NO: 3 - protein, 6 aa, Artificial Sequence
  Description of Artificial Sequence Synthetic peptide
QGIAGQ

SEQ ID NO: 4 - protein, 7 aa, Artificial Sequence
  Description of Artificial Sequence Synthetic peptide
QGIAGQR

SEQ ID NO: 5 - protein, 6 aa, Artificial Sequence
  Description of Artificial Sequence Synthetic peptide
FGIAGF

SEQ ID NO: 6 - protein, 5 aa, Artificial Sequence
  Description of Artificial Sequence Synthetic peptide
GIAGQ

SEQ ID NO: 7 - protein, 6 aa, Artificial Sequence
  Description of Artificial Sequence Synthetic peptide
QGAIAQ

SEQ ID NO: 8 - protein, 6 aa, Artificial Sequence
  Description of Artificial Sequence Synthetic peptide
FGIAGF

SEQ ID NO: 9 - protein, 6 aa, Artificial Sequence
  Description of Artificial Sequence Synthetic peptide
CGIAGC

SEQ ID NO: 10 - protein, 6 aa, Artificial Sequence
  Description of Artificial Sequence Synthetic peptide
EGIAGK

(19) United States
(12) Patent Application Publication
(10) Pub. No.: US 2007/0009568 A1
(43) Pub. Date: Jan. 11, 2007

(54) METHODS FOR IMMOBILIZING MOLECULES ON SURFACES

(76) Inventor: Rajendra S. Bhatnagar, Burlingame, CA (US)

Correspondence Address: WILSON SONSINI GOODRICH & ROSATI, 650 PAGE MILL ROAD, PALO ALTO, CA, 94304-1050, US

(21) Appl. No.: 11/422,224

(22) Filed: Jun. 5, 2006

Related U.S. Application Data
(60) Provisional application No. 60/687,295, filed on Jun. 3, 2005.

(51) Int. Cl. A61F 2/28; A61L 33/00

(52) U.S. Cl. 424/423; 427/2.24; 623/16.11

(57) ABSTRACT

The invention provides methods for immobilizing molecules on metallic surfaces, and compositions containing one or more surfaces that have been treated by these methods. The compositions may be used, e.g., to improve or repair cell, tissue, organ, or whole-body function.

### CROSS-REFERENCE

This claims benefit of Provisional Application No. 60/687,295, filed Jun. 3, 2005, which is incorporated by reference herein in its entirety.

### BACKGROUND OF THE INVENTION

Metals such as titanium, nickel, and aluminum in the form of pure metals or, more often, as components of alloys, are used for fabricating devices that are likely to come in contact with biological systems or that are placed in the body as implants in innumerable forms. It has been recognized for a long time that coating of such devices with a variety of organic molecules may increase their compatibility with biological systems. A variety of procedures have been used to coat metal surfaces. These range from plasma treatment and ion implantation to activate the surface to a variety of chemical procedures. However a need still remains for methods and compositions for coating surfaces, especially metal surfaces, to increase their compatibility with biological systems.

### SUMMARY OF THE INVENTION

The invention provides methods for treatment of a metal surface to modulate cell adherence to the surface, or to otherwise influence the behavior of cells and/or tissue exposed to the surface, and compositions that have been thus treated.

In one embodiment, the invention provides a method of preparing a surface comprising titanium, comprising contacting the surface with H₂0₂; reacting the surface with trifluoroethanesulfonyl chloride; and reacting the surface with a compound capable of influencing the behavior of cells when the surface is in contact with the cells. In some embodiments, the compound is capable of influencing the behavior of cells by promoting cell adherence to the surface or preventing cell adhesion to the surface. In some embodiments, the compound is capable of preventing cell adhesion to the surface. In some embodiments, the compound is a peptide containing a sequence of Gly-Thr-Pro-Gly-Pro-Gln-Gly-Ile-Ala-Gly-Gln-Arg-Gly-Val-Val; Gly-Pro-Gln-Gly-Ile-Ala-Gly-Gln-Arg; Gln-Gly-Ile-Ala-Gly-Gln; Gln-Gly-Ile-Ala-Gly-Gln-Arg; Phe-Gly-Ile-Ala-Gly-Phe; Gly-Ile-Ala-Gly-Gln; Gln-Gly-Ala-Ile-Ala-Gln; Phe-Gly-Ile-Ala-Gly-Phe; Cys-Gly-Ile-Ala-Gly-Cys; Glu-Gly-Ile-Ala-Gly-Lys; NAc-Ile-Ala-Ala; and Ile-Ala-βAla; or NAc-Ile-Ala-N-Me. In some embodiments the sequence contains the sequence G-T-P-G-P-Q-G-I-A-G-Q-R-G-V-V.

In another embodiment, the invention provides a composition comprising a surface prepared according to any of the methods described above, where the composition is a device for use in an animal to improve or repair cell, tissue, organ, or whole-body function. In some embodiments, the animal is a mammal, e.g., a human. In some embodiments, the device is used for improving joint function, repairing bone or soft tissue, providing plates for replacement or strengthening of bone or soft tissue, delivery of one or more drugs, improving cardiovascular function, improving urogenital function, providing an inlet or outlet for body fluids, or acting as a bioreactor. In some embodiments, the device is used for improving joint function; in some embodiments, the joint is hip, knee, elbow, finger, toe, ankle, vertebral, temporomandibular, or shoulder. In some embodiments, the joint is a hip joint.

In some embodiments, the joint is a knee joint. In some embodiments, the joint is a temperomandibular joint. In some embodiments, the device is used for improving cardiovascular function. In some of these embodiments, the device is a shunt or a stent. In some embodiments, the device is used for improving urogenital function. In some embodiments, the device is used for delivery of one or more drugs. In some embodiments, the device is used for providing an inlet or outlet for body fluids.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWING

FIG. 1 shows two micrographs that illustrate increased fibroblast binding to nitinol wire that was grafted with P-15 (bottom micrograph), a cell adhesion peptide, using the tresylation procedure, as compared with nitinol wire that was not grafted with P-15 (top micrograph).

The novel features of the invention are set forth with particularity in the appended claims. A better understanding of the features and advantages of the present invention will be obtained by reference to the following detailed description that sets forth illustrative embodiments, in which the principles of the invention are utilized.

### DETAILED DESCRIPTION OF THE INVENTION

The invention encompasses methods for stably immobilizing groups on surfaces to be exposed to biological environment, especially the surface of devices made from transition metals and their various alloys, as used in biomedical devices, prosthetics, analytical devices, and drug release systems. Groups that may be attached to the surface by the methods of the invention include drugs and pharmaceuticals, peptides, proteins, nucleic acids, proteoglyeans and various polymers and biopolymers. The methods of the invention modulate the biocompatibility of biomedical devices and allows the use of devices fabricated from metals such as titanium, aluminum, and nickel or alloys containing these metals to serve as hosts for biological molecules, cells, and as implants capable of integration with the host issue. In some embodiments, the methods may be used to attach groups to surfaces that discourage attachment or attraction of biological materials to the surface.

Any surface that will come in contact with a biological system and that is susceptible to the reactions described herein may be modified by the methods of the invention. The device and/or the surface to be treated by the methods of the invention may be flat, dense or of a complex shape. It may have a porous, beaded or meshed ingrowth surface. Surface materials include non-metals and metals. An exemplary non-metal is a polymer, e.g., a polysaccharide, such as methyl cellulose. Metallic surfaces include those typically found in medical implants, such as titanium, aluminum, nickel, tantalum, cobalt, chrome, niobium, molybdenum, zirconium, and stainless steel or alloys thereof. Devices that may be used in biological systems and that may include surfaces that can be treated by the methods of the invention include all of those presently used in the art and any others that may be made from materials compatible with the methods of the invention. Devices include, but are not limited to, orthopedic implants and prosthetic devices (e.g., joint replacements such as hip, knee, and the like, surgical screws, pins, plates, meshes used in reconstructive and other surgery, and dental implants), drug release systems (e.g., therapeutic pumps such as insulin pumps), stents, injection ports, electronic devices (e.g., pacemakers and analytical devices), and the like.

Such devices may be used as implants in the body, in humans and in animals for improving or assisting tissue and organ function (such as orthopedic implants including joints such as hip, knee, elbow, fingers etc), or repairing eye sockets or plates for implantation in the skull or other parts of the body, or devices for drug delivery, or shunts or stents and other devices in the cardiovascular system, and in the urogenital system, or inlets or outlets for body fluids, bioreactors and other devices.

The present invention is based on the observation that many elements, especially the metallic elements under consideration, are highly reactive in their surface layers. For example, the metallic elements described herein have a high affinity for oxygen and the ability to catalyze redox reactions with electron donors such as superoxide, free radical generators and electron donors such as H₂O₂, ascorbate, and dihydroxyfumarate, with generation of Metal-OH and Metal-OOH groups. The Metal-O bond is a facile anchorage for numerous reactive groups.

The methods of the invention generally proceed in three steps. First, the surface is treated to generate covalently attached —OH (hydroxyl) and/or —OOH (hydroperoxyl) groups. Second, the —OH or —OOH group is coupled to provide a reactive group appropriate for reaction with the group desired to be attached to the surface. Third, the reactive group is reacted with the group desired to be attached to the surface. In some embodiments, groups desired to be attached to the surface may be reacted directly with the —OH or —OOH group.

The first step may include cleaning the surface. For metals the surface may be cleaned of oil, grease and other contaminants. This may be done, e.g., by washing thoroughly in ethanol. The surface may then be washed with NaOH (e.g., 0.1 N NaOH) or soaked in an NaOH solution for, e.g., 15 minutes, followed by thorough washing with dI H2O. The surface is then treated with an electron donor. Exemplary electron donors include hydrogen peroxide, ascorbate, and dihydroxyfumarate and electrochemical processes. Other suitable electron donors will be apparent to those of skill in the art. In one embodiment, the surface is rinsed in full strength H2O2, then place in about 50% H2O2 overnight. Treatment of a surface, for example titanium, by the above method produces hydroxyl and hydroperoxyl groups bound to the surface atoms, e.g., Ti(OH)4 and TiOOH (hydroperoxide).

The second step is a coupling reaction. Any reagent that provides a reactive group that can couple covalently with a reactive moiety of the group desired to be attached to the surface and that can also be reacted to bond to the surface via the hydroxyl or hydroperoxyl group may be used. In some embodiments, the reagent may provide a group that can interact non-covalently with the desired moiety, or a group that can interact non-covalently with the hydroxyl and/or hydroperoxyl groups of the surface, or both.

In one embodiment, a facile leaving group is reacted with the hydroxy and/or hydroperoxy groups. An exemplary group is trifluoroethanesulfonyl chloride, which can be attached and provides a good leaving group (tresyl group) that can be displaced by a nucleophile, e.g., a peptide. Reaction conditions will be apparent to one of skill in the art; e.g. 200 ul trifluoroethanesulfonyl chloride may be combined with 1.0 ml pyridine and 20 ml tetrahydrofuran and reacted with the hydroxylated/hydroperoxylated surface.

In another embodiment, the hydroxylated/hydroperoxylated surface is reacted with a linking agent. A linking agent containing an oxidizable thiol and a side chain capable of forming a covalent link is used. Exemplary agents include cystamine, dimercaptosuccinic acid, 2-aminoethyl dithiocarbamate.

The third step in the methods is coupling of the desired group with the surface that has been prepared as above. If the surface has been treated to provide a good leaving group (e.g., by tresylation), then a nucleophile (e.g., a peptide) will displace the leaving group (e.g., a tresyl group) from surface OH groups and form a complex directly with the surface:
Surface-O-tresyl+peptide=Surface-O-peptide+reactants

If a thiol group has been attached to the surface, then following the grafting of the thiol compound that donates an electron to existing oxide or hydroxide groups, the side chain can be bonded to the group to be attached using appropriate reaction chemistry, as will be apparent to one of skill in the art. For example, a peptide or protein can be covalently bonded to the side chain with a variety of bond forming reactions, including the well known peptide bond formation reaction carried out in the presence of 1-ethyl-3-(3-dimethylaminopropyl)-carbodiimide (“water soluble carbodiimide”) and N-hydroxysuccinimide. Other biologically active compounds, for example drugs, and polymers carrying reactive groups such as —NH2,—SH, —COO, may be bonded in a similar manner

In some embodiments, the group to be bonded to the surface is reacted directly with the hydroxyl/hydroperoxyl groups on the surface. The group may be modified to provide reactive sites for this reaction, or may naturally contain such sites and be reacted in unmodified form. For example, a peptide to be immobilized on a redox-modified surface may be extended to incorporate an electron donor moiety such as a thiol, or a carboxyl rich amino acid. This modified peptide is directly reacted with the surface following treatment of latter as described above. Alternatively, naturally-occurring thiols or carboxyl side chains of amino acids of the peptide may be reacted with the surface following treatment of the latter as described above.

In one embodiment, the surface is a metal, preferably titanium, and the group to be attached is a peptide, e.g., P-15 peptide. The procedure in this embodiment includes washing the surface thoroughly in ethanol, then soaking in a 0.1 N NaOH solution for 15 minutes, followed by washing 10× with DI H2O. Then the surface is rinsed in full strength H2O2, and place in 50% H2O2 overnight. The surface is then exposed to a tresylation reaction mixture of trifluoroethanesulfonyl chloride (200 ul), pyridine (1.0 ml), and tetrahydrofuran (20 ml). After reaction and washing, the surface is then exposed to the desired peptide (e.g., P-15) and the peptide displaces the tresyl group to form a covalent linkage with the metal surface. See Example 1.

In some embodiments, the treated surface is linked to a group, e.g., a bioactive molecule, that promotes tissue growth, stabilization and/or integration onto the surface. In some embodiments, the treated surface is linked to groups that prevent or retard cell and/or tissue adherence, migration, and/or integration onto the surface. The latter embodiment is useful for applications in which it is desired to keep surfaces such as apertures free of contamination, e.g., in implantable injection ports. It will be apparent to the skilled artisan that various surfaces in a device may be linked to groups to promote cell and/or tissue migration and adherence whereas other surfaces in the same device may be linked to groups that prevent such processes.

Groups that may be attached to a surface using the methods of the invention include peptide, protein, nucleic acid, proteoglycans, polymer, biopolymer, drug, pharmnaceutical, antibody, cell, monosaccharide, oligosaccharide, polysaccharide, lipid, inorganic or organic molecule, or combinations thereof For promotion of cell and/or tissue migration, growth, and adherence, useful groups include growth factors, attachment factors, drugs, and the like. It is especially desirable to attach a group or groups to the surface that will promote its integration and compatibility with the tissue to which the surface is exposed, e.g., the major tissue to which the surface is exposed is bone for joint replacements, screws, nails, plates, and the like, whereas the major tissue to which the surface is exposed is soft tissue such as connective tissue and muscle for, e.g., injection ports. It will be appreciated that a device may contain surfaces exposed to various tissues, and the group or groups attached to the surface can be selected and arranged according to the likely tissue exposure of the particular area of the surface to be treated. Groups useful in the methods of the invention include normal and/or derivative osteopontin, bone sialoprotein, bone acidic glycoprotein-75, osteocalcin, osteonectin, bone morphogenetic proteins, transforming growth factors and transforming growth factor mimics (see, e.g., U.S. Pat. No. 5,661,127; 5,780,436; and 6,638,912, all of which are incorporated by reference herein in their entirety), laminin, type IV collagen, type VIII collagen, enamel proteins (amelogenins and non-amelogenins), α2 HS-glycoprotein, fibronectin, cell adhesion peptides, prostaglandin, serum proteins, glucocorticosteroids (e.g., dexamethasone), phosphate, phosphoserine, pyrophosphates, phosphothreonine, phosvitin, phosphophdryn, biphosphonates, phosphonates, phosphatases, sulfonates, sulfates, carboxy group, bone and epithelial proteoglycans, polyaspartate, and other biological molecules capable of promoting tissue integration, and non-biological molecules chosen to imitate these effects. In some embodiments the group is an antibody to growth factors, which binds normal growth factors in body fluids and immobilizes them at the surface of, e.g., a surgical implant (such as a hip implant), thus promoting growth at the implant site. See U.S. Pat. No. 4,828,563, which is incorporated by reference herein in its entirety. In some embodiments, the group is mineral and cell binding peptide sequences such as Arginine-Glycine-Aspartic acid (Arg-Gly-Asp); see U.S. Pat. No. 6,291,428, which is incorporated by reference herein in its entirety, for a further description of related peptides.

In some embodiments the group is a collagen analog of the cell-binding domain, which promotes migration and adherence of soft tissue cells such as fibroblasts and endothelial cells. Collagen analogs suitable for practicing the present invention are described in U.S. Pat. Nos. 6,638,912; 5,354,736; and 5,635,482 (all to Bhatnagar), all of which are incorporated herein by reference in their entirety.

A particularly preferred collagen receptor agonist is sometimes called “P-15” and has the sequence G-T-P-G-P-Q-G-I-A-G-Q-R-G-V-V. Other useful collagen analogs, described in U.S. Pat. Nos. 6,638,912; 5,354,736; and 5,635,482, include Gly-Pro-Gln-Gly-Ile-Ala-Gly-Gln-Arg; Gln-Gly-Ile-Ala-Gly-Gln; Gln-Gly-Ile-Ala-Gly-Gln-Arg; Phe-Gly-Ile-Ala-Gly-Phe; Gly-Ile-Ala-Gly-Gln; Gln-Gly-Ala-Ile-Ala-Gln; Phe-Gly-Ile-Ala-Gly-Phe; Cys-Gly-Ile-Ala-Gly-Cys; Glu-Gly-Ile-Ala-Gly-Lys; NAc-Ile-Ala-Ala; and Ile-Ala-βAla; and NAc-Ile-Ala-N-Me.

The P-15 region represents half of one turn of the collagen triple helix, i.e. fifteen residues, which is believed to be exposed in intact collagen molecules on the surface of fibers. The other half of the turn faces the core of the fiber. Theoretical and experimental studies showed that the sequence contained in P-15 can acquire a conformation dramatically different from the triple helical conformation generally observed in the rest of the collagen molecule. This atypical, or “non-collagen,” conformation is believed necessary for recognition by the docking of collagen binding species, such as cell surface receptors for collagen and fibronectin. The three dimensional surface presented by the P-15 region or parts of the P-15 region is complementary to the reactive surface present on the binding species (receptors, fibronectin).

In cases where it is desired to minimize or prevent cell-cell contact and tissue adhesion, groups such as those described in U.S. Pat. No. 6,743,521, incorporated by reference herein in its entirety, may be used.

The invention also provides compositions wherein a transition metal surface is bonded to a group as described herein. In some embodiments, the composition includes a titanium surface that is covalently bonded to a P-15 group; such a titanium surface may be, e.g., a surface of an implant, such as a dental implant or orthopedic implant. Thus, in some embodiments the invention provides a composition that has at least one surface prepared according to the methods of the invention, where the composition is a device for use in an animal to improve or repair cell, tissue, organ, or whole-body function. In some embodiments the animal is a mammal; in some embodiments the animal is a human. Non-exclusive examples of purposes for which the device can be used include improving joint function, repairing bone or soft tissue, providing plates for replacement or strengthening of bone or soft tissue, delivery of one or more drugs, improving cardiovascular function, improving urogenital function, providing an inlet or outlet for body fluids, or acting as a bioreactor. When the device is used for improving joint function, examples of joints in which the device may be used include, but are not limited to, hip, knee, elbow, finger, toe, ankle, vertebral, temporomandibular, and shoulder. In some embodiments, the device is used for improving cardiovascular function, and the device is a shunt or a stent.

### EXAMPLES

### Example 1

### Tresylation Followed by Nucleophilic Displacement with P-15

In this Example, titanium wire was treated with tresylation and attachment of P-15, followed by exposure to fibroblasts. The titanium wire was washed thoroughly in ethanol, then soaking in a 0.1 N NaOH solution for 15 minutes, followed by washing 10× with DI H2O. The surface was then rinsed in full strength H2O2, then soaked in 50% H₂O₂ overnight. The wire was then placed in a tresylation reaction mixture of trifluoroethanesulfonyl chloride (200 ul), pyridine (1.0 ml), and tetrahydrofuran (20 ml) overnight, followed by washing in DI H₂O. The surface was then placed in a solution containing P-15 1 mg/ml in 100 mM phosphate buffer, pH 7.4, overnight. Following this treatment, the wire was again rinsed in DI H₂O, then placed in a cell culture which contained fibroblasts that had been fluorescently labeled for 24 hours.

As a control, a second titanium wire was treated identically to the first wire, described above, except that it was not exposed to P-15 before being exposed to the fluorescently-labeled cells.

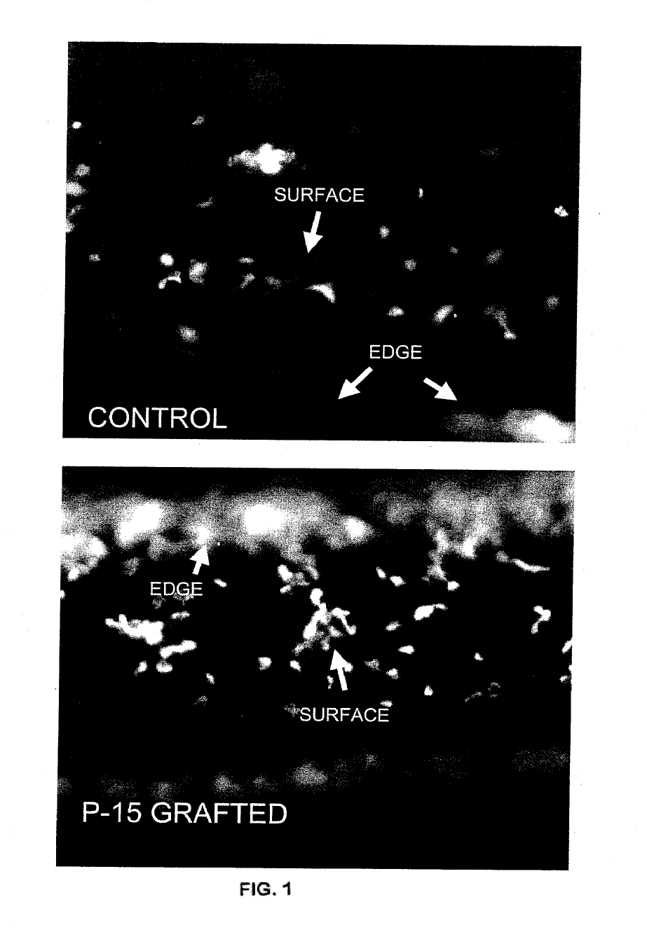

FIG. 1 presents two photomicrographs that show the results of this procedure. The upper photomicrograph depicts the control wire. The surface of the wire, which is in the focal plane, has few fluorescently labeled cells attached. The edge of the wire, which is out of the focal plane and thus somewhat blurred, also has few labeled cells attached. In contrast, the wire that was treated with P-15 shows far more attached cells both on the surface and edges, as seen in the bottom photomicrograph of FIG. 1.

This Example demonstrates that tresylation of a titanium surface followed by treatment with P-15 results in the attachment of fibroblasts to the surface when the surface is exposed to the cells.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of preparing a surface comprising titanium, comprising
(a) contacting the surface with H₂O₂;
(b) reacting the surface with trifluoroethanesulfonyl chloride; and
(c) reacting the surface with a compound capable of influencing the behavior of cells when the surface is in contact with the cells.

2. The method of claim 1 wherein the compound is capable of influencing the behavior of cells by promoting cell adherence to the surface or preventing cell adhesion to the surface.

3. The method of claim 2 wherein the compound is capable of promoting cell adherence to the surface.

4. The method of claim 2 wherein the compound is capable of preventing cell adhesion to the surface.

5. The method of claim 1 wherein the compound is a peptide comprising a sequence selected from the group consisting of Gly-Thr-Pro-Gly-Pro-Gln-Gly-Ile-Ala-Gly-Gln-Arg-Gly-Val-Val (SEQ ID NO: 1); Gly-Pro-Gln-Gly-Ile-Ala-Gly-Gln-Arg (SEQ ID NO: 2); Gln-Gly-Ile-Ala-Gly-Gln (SEQ ID NO: 3); Gln-Gly-Ile-Ala-Gly-Gln-Arg (SEQ ID NO: 4); Phe-Gly-Ile-Ala-Gly-Phe (SEQ ID NO: 5); Gly-Ile-Ala-Gly-Gln (SEQ ID NO: 6); Gln-Gly-Ala-Ile-Ala-Gln (SEQ ID NO: 7); Phe-Gly-Ile-Ala-Gly-Phe (SEQ ID NO: 8); Cys-Gly-Ile-Ala-Gly-Cys (SEQ ID NO: 9); Glu-Gly-Ile-Ala-Gly-Lys (SEQ ID NO: 10); NAc-Ile-Ala-Ala; and Ile-Ala-βAla; and NAc-Ile-Ala-N-Me.

6. The method of claim 1 wherein the compound is a peptide comprising the sequence G-T-P-G-P-Q-G-I-A-G-Q-R-G-V-V (SEQ ID NO: 1).

7. A composition comprising a surface prepared according to the method of claim 1, wherein the composition is a device for use in an animal to improve or repair cell, tissue, organ, or whole-body function.

8. The composition of claim 7 wherein the animal is a mammal.

9. The composition of claim 7 wherein the animal is a human.

10. The composition of claim 7, wherein the device is used for a purpose selected from the group consisting of improving joint function, repairing bone or soft tissue, providing plates for replacement or strengthening of bone or soft tissue, delivery of one or more drugs, improving cardiovascular function, improving urogenital function, providing an inlet or outlet for body fluids, and acting as a bioreactor.

11. The composition of claim 10, wherein the device is used for improving joint function

12. The composition of claim 11 wherein the joint is selected from the group consisting of hip, knee, elbow, finger, toe, ankle, vertebral, temporomandibular, and shoulder.

13. The composition of claim 12 wherein the joint is a hip joint.

14. The composition of claim 12 wherein the joint is a knee joint.

15. The composition of claim 12 wherein the joint is a temperomandibular joint.

16. The composition of claim 10, wherein the device is used for improving cardiovascular function.

17. The composition of claim 16 wherein the device is a shunt or a stent.

18. The compostion of claim 10 wherein the device is used for improving urogenital function.

19. The composition of claim 10 wherein the device is used for delivery of one or more drugs.

20. The composition of claim 10 wherein the device is used for providing an inlet or outlet for body fluids.
